(19) United States
(12) Patent Application Publication
(10) Pub. No.: US 2009/0048679 A1
(43) Pub. Date: Feb. 19, 2009

(54) IMPLANT

(75) Inventors: Ralph Howald, Gossau (CH); Peter Heuberger, Raterschen (CH); Ulrike Trommsdorff, Zurich (CH)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER GMBH, Winterthur (CH)

(21) Appl. No.: 12/278,835

(22) PCT Filed: Feb. 2, 2007
(86) PCT No.: PCT/EP07/51008

§ 371 Date: Sep. 17, 2008

(30) Foreign Application Priority Data
Feb. 9, 2006 (CH) 00207/06

(51) Int. Cl. A61F 2/40; A61F 2/28

(52) U.S. Cl. 623/19.12; 623/16.11

(57) ABSTRACT

The invention relates to an implant for the relief of damaged regions of the joint surfaces of hip or shoulder joints which may be introduced into the gap between the joint surfaces of the joint head and the joint socket in the natural joint, the implant being embodied as a shell or cap which is laid in the implanted state with a convex outer surface in the joint socket and a convex inner surface sitting on the joint head. The implant has a body section forming the shell or cap with an opening and a boundary section defining the opening and the implant is made from a flexible material which may be deformed for introduction of the implant into the gap between the joint surfaces.

The invention relates to an implant for the relief of damaged regions of the joint surfaces of hip joints or shoulder joints.

In known joint prostheses, both joint surfaces are usually replaced by mutually matched artificial joint parts. As is known, this is done within the framework of surgery in which a more or less large portion of the healthy bone is removed in dependence on the respective prosthesis used. In hip operations, for example, the femoral head is fully or partly removed to make room for a joint head which is anchored in the femur via a shaft. Accordingly, bone material is removed at the acetabulum of the hip bone by corresponding machining during the surgery to be able to apply an artificial joint socket or joint shell which serves for the reception of the joint head.

Such operations are associated with strains and risks which are not insignificant for the patient. It is particularly problematic with relatively young patients that a problem-free function of an artificial joint cannot be guaranteed over periods of any desired length. Follow-up operations are necessary in many cases. Such revisions are, however, frequently difficult to carry out since the first operation already costs the patient a frequently not insubstantial portion of the healthy bone material.

The first operation, which results in a relevant loss of bone material, can be delayed by so-called resurfacing techniques. In this respect, only the surfaces of the affected joints are replaced by comparatively thin implants of cap shape or cup shape. These known techniques, however, also require operations which are a strain and which are likewise not possible in a minimally invasive manner. It is furthermore also necessary in resurfacing to remove healthy bone material in the region of the affected joint surfaces, albeit to a lesser degree than in the previously mentioned operations.

These considerations are above all of significance when the joint surfaces are affected by arthrosis and the affected bones themselves are intact per se, i.e. no fractures have to be treated. Such joint damage also occurs in relatively young patients so that there is a need for techniques for the treatment of damaged joint surfaces in which the healthy bone material should be maintained as far as possible. To keep the strain for the patient as small as possible, such techniques should be able to be carried out in a minimally invasive manner.

It is the aim of the invention to provide an implant which satisfies these demands.

The invention provides an implant for the relief of damaged regions of the joint surfaces which can be introduced into the gap between the joint surfaces of the joint head and the joint socket cooperating in the natural joint, wherein the implant is made as a shell or as a cap whose convex outer side is disposed in the joint socket and whose concave inner side is seated on the joint head in the implanted state, wherein the implant has a body section which forms the shell or cap and has an opening and a marginal section which bounds the opening, and wherein the implant is manufactured from a flexible material which can be deformed for the introduction of the implant into the gap present between the joint surfaces.

The implant can considerably reduce compressive strain and/or shear strain of regions of the joint surfaces affected by arthrosis. The implant is thereby not exclusively suitable for joints affected by arthrosis in the early stage, but is particularly well suited therefor.

The time at which a first operation of the initially described kind becomes inevitable can be postponed by the implant. The implant is therefore not exclusively interesting for relatively young patients, but is particularly good for them.

It is of advantage that the implant can be used within the framework of a minimally invasive procedure. No machining of the bone is required. The implant can thus be made such that is aligns itself in the joint gap. Alternatively, the implant can be fixed at the natural bone structure or tissue structure.

It is furthermore of advantage that the use of the implant does not require any subluxation of the joint head.

It has been found that the natural joint function can be maintained by a suitable material and/or by a suitable material coating for the implant.

The implant can be manufactured from one or more different metallic or non-metallic materials. A hydrogel, polyurethane or polyethylene can be considered, for example.

The implant can include a textile material or consist of a textile material, with the textile material including a textile substrate and with a coating being provided at at least one part of the surface of the textile substrate which includes a compound selected from the group consisting of hydrogels, polyurethanes, polyvinyl chloride, polytetrafluoroethylene and any desired combinations of two or more of the aforesaid compounds.

The textile substrate can consist of fibers, with at least some of the fibers being provided with a coating which includes a compound selected from the group consisting of hydrogels, polyurethanes, polyvinyl chloride, polytetrafluoroethylene and any desired combinations of two or more of the aforesaid compounds.

Sliding properties can thereby be realized which at least largely correspond to the natural conditions in the joint.

The implant can furthermore have a sandwich structure of a plurality of individual layers. At least two layers can differ from one another with respect to their structure and/or their material.

The implant, or at least one layer of a sandwich structure of the implant, which includes a plurality of individual layers can be provided with a reinforcement of textile, fiber, woven fabric, knitted fabric or fabric.

Two adjacent layers having the same material, but a different structure, can be chemically cross-linked with one another, with the cross-linking having been generated by irradiation, for example.

The material of the implant can be bioresorbable.

Provision can furthermore be made for the implant to include a plurality of relief members each forming a part region of the outer side and/or of the inner side. The relief members can be made as pads, cushions or balls. The relief members can be connected to one another by an areal carrier structure. The carrier structure can include a textile material, fiber material, woven fabric material, knitted fabric material or fabric material.

The marginal section can be provided with a reinforcement. Separate strength carriers can be integrated into the marginal section. A reinforcement of the marginal section can take place by a fiber structure or by a cord structure.

It is also possible for the marginal section to be hollow and to be provided with a filling. The filling can, be a gas, for example air, or a liquid. A filling material can be provided whose state changes over time. While the filling material is liquid or pasty on the insertion of the implant to facilitate the insertion, the filling material changes into a solid state in the course of time so that the marginal section gives the implant a high shape stability and/or provides a stable positioning of the implant between the two joint surfaces.

The body section of the implant can include a spherical section and a cylindrical section, with the cylindrical section adjoining the spherical section. The spherical section can have the shape of a semi-sphere.

The marginal section can the thickened with respect to the wall thickness of the body section. Provision can be made in this respect for the marginal section to be made in bead shape.

The implant can be dimensioned in dependence on the respective joint such that, in the implanted state, the marginal section is disposed outside the gap present between the joint surfaces.

To facilitate a deformation of the implant on the insertion and/or to take the anatomy of the respective joint into account, the body section of the implant can have at least one cut-out. On insertion at the hip, this cut-out can serve to maintain the so-called ligamentum capitis femoris.

The cut-out can extend up to and into the marginal section, with the marginal section being able to have an interruption formed by the cut-out. A separate adjustment piece can be provided which can be inserted into the interruption of the marginal section after the insertion of the implant into the gap between the joint surfaces to close the marginal section. The adjustment piece can thus give the implant shape stability again in the inserted state.

It is possible, but not compulsory, for the implant to have a constant wall thickness. If a cut-out of the kind explained above is provided, the wall thickness of the body section can vary such that the wall thickness close to the cut-out is larger than in the regions disposed further away from the cut-out.

In a possible embodiment, a wall thickness distribution results in that, starting from a symmetrical state in which the body section has a constant wall thickness between the inner side and the outer side, the inner side and the outer side are displaced with respect to one another. This offset can take place in a direction which extends parallel to an equatorial plane of a spherical section.

If a cut-out of the kind explained above is provided, the offset of the inner side relative to the outer side can then take place in a direction away from the cut-out. A distribution of the kind already mentioned above hereby results, according to which the wall thickness is larger in the region of the cut-out than in the regions remote from the cut-out.

Further possible embodiments result from the dependent claims, from the description and from the drawing.

The invention will be described in the following by way of example with reference to the drawing. There are shown:

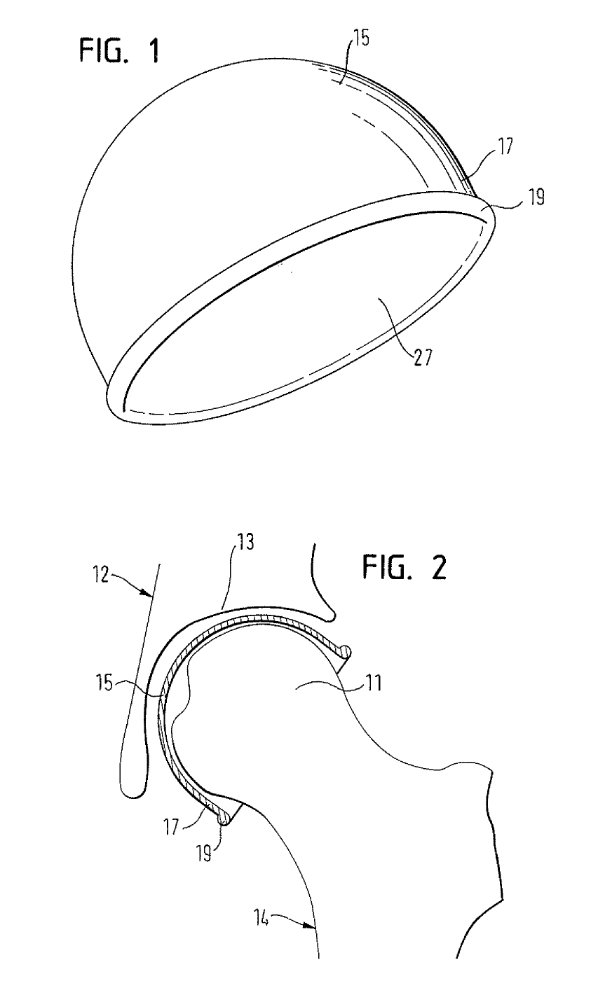

FIGS. 1-3 an embodiment of an implant in accordance with the invention, and

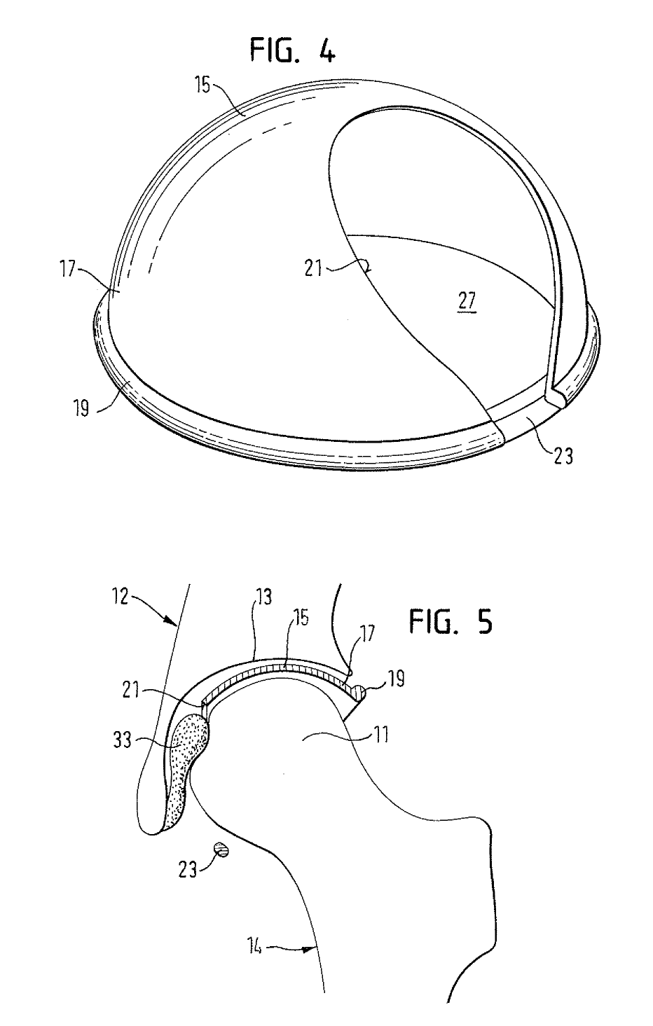

FIGS. 4-7 a further embodiment of an implant in accordance with the invention.

The implant can be used both for hip joints and for shoulder joints. Two embodiments are explained in the following, and indeed only in conjunction with a hip joint, for example.

The implant in accordance with FIGS. 1 to 3 is made as a cap which comprises a spherical section 15 in the form of a semi-sphere. A cylindrical section 17 adjoins the spherical section 15.

The cap is open to the bottom. The opening 27 is bounded by a marginal section 19. The marginal section 19 comprises a bead-shaped thickened portion such that the marginal section 19 projects radially outwardly with respect to the cylindrical section 17.

The insertion of the implant can take place within the framework of an arthroscopic procedure. The joint surfaces can be separated, for example, e.g. by use of an extraction table, and indeed without subluxation of the femoral head 11. The insertion of the implant can also take place by other minimally invasive procedures.

The implant consists of a flexible material. The implant can hereby be deformed on insertion and can adjust to the shape of the joint gap between the femoral head 11 and the joint socket 13. Reference is also made to the introductory part with respect to the possible materials or the possible structure of the implant.

The material can be metallic or non-metallic. A hydrogel, polyurethane or polyethylene can be considered, for example. The material can be reinforced by fiber structures or canvas structures. The material can furthermore consist of a plurality of coatings or layers which are manufactured either from different materials or from the same material with different structures. The implant material can comprise a substrate and a substrate coating. The substrate can be a textile material.

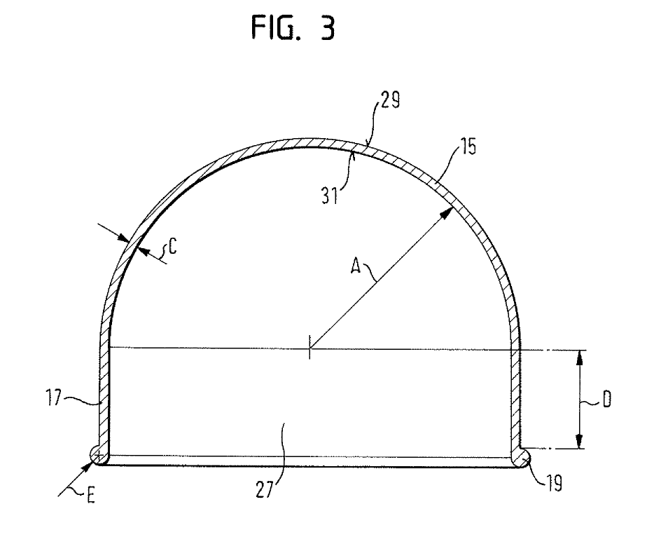

With reference to FIG. 3, the inner radius A of the spherical section and of the cylindrical section can lie, for example, in the range from 38-60 mm. The length D of the cylindrical section 17 amounts, for example, to half or less of the inner radius A. The cylindrical section 17 can be as short as desired, with a cylindrical section 17 also being able to be dispensed with completely.

The wall thickness C of the implant is constant and amounts, for example, to 0.5 to 3 mm.

The bead-shaped marginal section 19 is at least approximately circular in cross-section. The radius E of the bead is, for example, in the range from 0.5 to 4.0 times the wall thickness C.

The bead-shaped marginal section 19 can be manufactured in solid form and completely from the same material as the body section formed by the spherical section 15 and by the cylindrical section 17. Alternatively, the marginal section 19 can be reinforced, for example, by a cord structure. It is also possible to make the marginal section 19 hollow and to fill it with a gas, a liquid or a material changing in the course of time from a liquid or pasty starting state into a solid end state, as is explained in the introductory part.

With a corresponding embodiment, the bead-shaped marginal section 19 can ensure that the implant aligns itself in the inserted state.

To prevent relative movements between the implant and the joint socket 13 on the movement of the joint or to reduce the magnitude of these movements, the implant can be fastened to the marginal region of the acetabulum, and indeed to the bone or to the fibrous cartilage-like material of the labrum acetabuli.

It is also possible to fasten the marginal section 19 of the implant to the neck of the femur, with a cord reinforcement of the marginal section 19 optionally provided being able to be used. An articulation of the implant at the femur side can hereby be prevented or its magnitude reduced.

The embodiment in accordance with FIGS. 4 to 7 differs from the embodiment in accordance with FIGS. 1 to 3 by a cut-out 21 provided in the body section 15, 17 and by a non-constant wall thickness.

The cut-out 21 extends from the spherical section 15 over the cylindrical section 17 up to and into the marginal section 19. The marginal section 19 is interrupted on the basis of the cut-out 21. To close the interruption again after the insertion of the implant and to restore the shape stability of the implant, a separate adjustment piece 23 is provided.

The adjustment piece 23 is removed on the insertion of the implant. The implant is pushed, starting from the cranial margin of the acetabulum 13, between the joint surfaces of the femoral head 11 and of the hip bone 12. In this respect, the flexible implant adapts to the shape of the joint surfaces on its own. As already, mentioned, the ligamentum capitis femoris 33, which is disposed in the region of the cut-out 21 in the implanted state (FIG. 5), is maintained due to the cut-out 21.

The marginal section 19 can then be closed by the adjustment piece 23. The implant hereby again attains a shape stability in accordance with the embodiment without a cut-out in accordance with FIGS. 1 to 3.

A slight asymmetry in the distribution of the wall thicknesses, such as is described in the following, gives the implant rotational stability. Stability against rotation can also be achieved in that the marginal section 19 of the implant is fixed to the marginal region of the acetabulum 13.

Reference is made to the explanations on the embodiment in accordance with FIGS. 1 to 3 with respect to the material and to the structure of the implant.

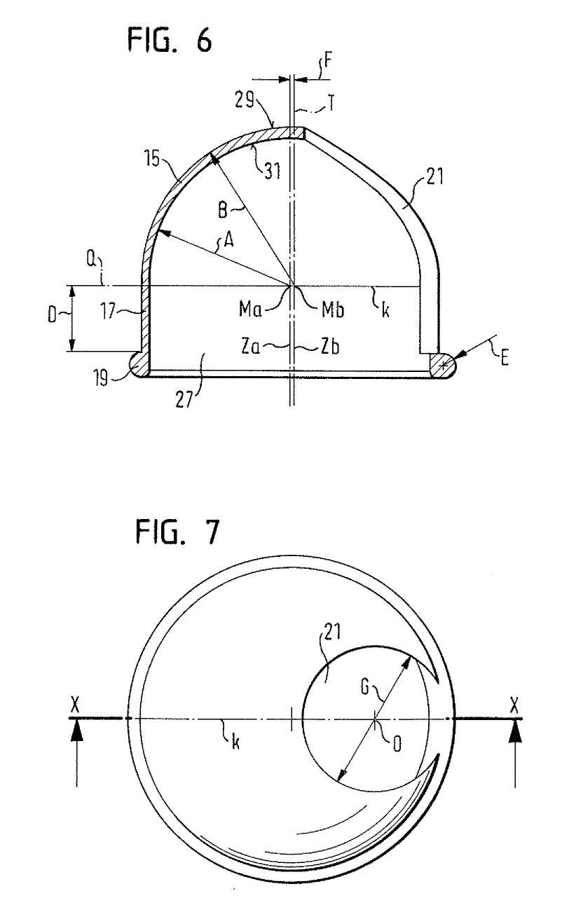

The irregular wall thickness distribution of the implant arises in that (FIG. 6) the outer side 29 and the inner side 31, starting from a symmetrical state with a constant wall thickness, are displaced against one another, and indeed along a line k which extends parallel to an equatorial plane Q. In other words, the outer side 29 and the inner side 31 form two spherical part surfaces which are disposed eccentrically in one another. The direction of the displacement is selected such that the inner side 31 is displaced away from the cut-out 21. The center Ma of the inner side 31 is thus disposed at the side of the center Mb of the outer side remote from the cut-out 21.

In this respect, these centers Ma, Mb and the center O of a circular region of a projection of the cut-out 21 onto the equatorial plane Q are disposed on a line k (FIG. 7). The circular region with a center O has a diameter G which can generally be as desired as long as the cut-out 21 does not extend up to the pole of the implant (FIG. 6). Two half-spaces can thus be defined which are separated from one another by a plane T in which the center Mb of the outer side 29 is disposed through which the central axis Zb of the corresponding outer surface of the cylindrical section 17 extends. The cut-out 21 is located in the one half-space, whereas the center Ma of the inner side 31 and the central axis Za of the corresponding inner surface of the cylindrical section 17 are disposed in the other half-space.

The magnitude of the inner radius A and the length D of the cylindrical section 17 can be dimensioned according to the embodiment in accordance with FIGS. 1 to 3d. The difference between the outer radius B and the inner radius A lies, for example, in the range from 0.5 to 3 mm. The radius E of the bead-shaped marginal section 19 lies, for example, in the range of 0.5 to 3.0 times the aforesaid difference between the outer radius B and the inner radius A.

The offset F between the two centers Ma and Mb, and thus between the central axes Za, Zb, can amount up to 2.5 mm.

The larger wall thickness in the region of the cut-out 21, and thus in the caudal region of the acetabulum, results due to the direction of the offset F along the line k explained above.

Alternatively to the materials and to the structure of the implant, such as was explained initially and above in conjunction with the embodiment in accordance with FIGS. 1 to 3, the implant can consist of a plurality of soft individual elements which are made in the manner of pads, cushions or balls and can serve as relief members. The relief members define the contact surfaces of the implant to the joint surfaces of the femoral head 11 and of the acetabulum 13 and thus serve for the relief of already damaged regions of the joint surfaces. The relief members are connected to one another by an areal carrier structure which can consist of a metallic or non-metallic material. The carrier structure can include a textile material, fiber material, woven fabric material, knitted fabric material or fabric material.

In each of the embodiments described, the invention provides an implant by which damaged regions of the joint surfaces can be relieved. This relief can have the effect that the damaged regions recover again. Operations which are a strain on the patient and signify a loss of healthy bone material can be delayed in this manner. As already initially explained, this is above all of advantage for relatively young patients.

### REFERENCE NUMERAL LIST

11 joint head

12 hip bone

13 joint socket

14 femur

15 spherical section

17 cylindrical section

19 marginal section

21 cut-out

23 adjustment piece

27 opening

29 outer side

31 inner side

33 ligamentum capitis femoris

A inner radius

B outer radius

C wall thickness of the body section

D length of the cylindrical section

E radius of the bead-shaped marginal section

F offset

G diameter of the circular region of the cut-out

O center of the circular region of the cut-out

Q equatorial plane

T plane

Ma center of the inner side

Mb center of the outer side

Za central axis of the inner surface of the cylindrical section

Zb central axis of the outer surface of the cylindrical section

k connection line between Ma and Mb

1-27. (canceled)

28. An implant for the relief of damaged regions of the joint surfaces, in particular of hip joints or shoulder joints, which can be introduced into the gap between the joint surfaces of the joint head and the joint socket cooperating in the natural joint, the implant comprising:
a shell having a convex outer side sized to be in the joint socket and a concave inner side is sized to be seated on the joint head in the implanted state,
wherein the implant has a body section which forms the shell and has an opening and a marginal section which bounds the opening, and
wherein the implant is manufactured from a flexible material which can be deformed for the introduction of the implant into the gap present between the joint surfaces.

29. An implant in accordance with claim 28, wherein the implant is manufactured from one or more different non-metallic materials, for example from a hydrogel, polyurethane or polyethylene.

30. An implant in accordance with claim 28, wherein the implant includes a textile material or consists of a textile material, wherein the textile material includes a textile substrate and wherein a coating is provided at at least one part of the surface of the textile substrate, said coating including a compound selected from the group consisting of hydrogels, polyurethanes, polyvinyl chloride, polytetrafluoroethylene and any desired combinations of two or more of the aforesaid compounds.

31. An implant in accordance with claim 28, wherein the implant includes a textile material or consists of a textile material, wherein the textile material includes a textile substrate and wherein the textile substrate consists of fibers and at least some of the fibers are provided with a coating which includes a compound selected from the group consisting of hydrogels, polyurethanes, polyvinyl chloride, polytetrafluoroethylene and any desired combinations of two or more of the aforesaid compounds.

32. An implant in accordance with claim 28, wherein the implant has a sandwich structure of a plurality of individual layers.

33. An implant in accordance with claim 28, wherein the implant or at least one layer of a sandwich structure of the implant having a plurality of individual layers is provided with a reinforcement of textile, fiber, woven fabric, knitted fabric or fabric.

34. An implant in accordance with claim 32, wherein two adjacent layers having the same material but a different structure, are mutually chemically cross-linked, with the cross-linking having been generated by irradiation, for example.

35. An implant in accordance with claim 28, wherein the marginal section is provided with a reinforcement.

36. An implant in accordance with claim 28, wherein the body section includes a spherical section and a cylindrical section which adjoins the spherical section.

37. An implant in accordance with claim 28, wherein the marginal section is designed such that it gives the body section shape stability in the implanted state.

38. An implant in accordance with claim 28, wherein the marginal section is thickened with respect to the wall thickness C of the body section.

39. An implant in accordance with claim 28, wherein the body section has a cut-out, preferably wherein the cut-out extends up to and into the marginal section and the marginal section has an interruption formed by the cut-out.

40. An implant in accordance with claim 39, wherein a separate adjustment piece is provided which can be inserted into the interruption of the marginal section after the introduction of the implant into the gap between the joint surfaces to close the marginal section.

41. An implant in accordance with claim 39, wherein the wall thickness of the body section close to the cut-out is larger than in regions disposed further away from the cut-out.

42. An implant in accordance with claim 28, wherein, starting from a symmetrical state in which the body section has a constant wall thickness between the inner side and the outer side, the inner side and the outer side are displaced with respect to one another, preferably wherein the implant has a cut-out and the inner side is displaced in a direction away from the cut-out.
